(19) United States
(12) Patent Application Publication
(10) Pub. No.: US 2022/0381758 A1
(43) Pub. Date: Dec. 1, 2022

(54) SYSTEMS AND METHODS FOR SAMPLING CHEMICAL SPECIES IN VARIOUS ENVIRONMENTS

(71) Applicant: Chevron U.S.A. Inc., San Ramon, CA (US)

(72) Inventors: David Glynn Thomas, Perth (AU); Paul Bireta, Houston, TX (US); Travis Samuel Elsdon, Perth (AU); Michael John Marnane, Perth (AU)

(21) Appl. No.: 17/618,663

(22) PCT Filed: Jun. 12, 2020
(86) PCT No.: PCT/US20/37382

§ 371 Date: Dec. 13, 2021

Related U.S. Application Data
(60) Provisional application No. 62/861,728, filed on Jun. 14, 2019.

(51) Int. Cl. G01N 33/18; G01N 1/02; G01N 1/40

(52) U.S. Cl. CPC G01N 33/18; G01N 1/02; G01N 1/405; G01N 2001/021

(57) ABSTRACT

A cartridge sampler for collecting a target species from an environment for subsequent chemical analysis includes removable collection media having at least one molecule sensor thereon. The cartridge sampler includes a means for tagging a geospatial location and time that the fluid samples are provided to the collection media such that the location and time that the fluid samples are provided to the collection media can be later determined. Also disclosed are systems and methods for collecting species from various environments including a subsurface formation, a pipeline, and gaseous, soil or sub-slab, and aqueous environments. The cartridge sampler is fitted on or within a device designed for moving within each environment.

### FIELD

The disclosure relates to the field of sampling systems and sensors for collecting and detecting chemical species in various environments.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Techniques are known to collect data relating to underwater hydrocarbon reserves or underwater infrastructure transporting fluid that contains hydrocarbons. For instance, reflection seismology or non-seismic detection technologies such as magnetometers are typically used. Satellite and airborne imaging, or ship-borne multibeam imaging together with drop core sampling are often used. To gather data closer to the seabed, tethered or untethered remotely operated vehicles (ROV's) can be used. A cartridge sensor for sampling and sensing hydrocarbons in a subsea environment in an autonomous underwater vehicle (AUV) can be used, as disclosed in U.S. Pat. No. 9,404,906 B2 (Thomas et al.).

There exists a need for additional, cost effective sampling systems and sensors for collecting and detecting a variety of chemical species in a variety of environments.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In general, in one aspect, the disclosure relates to a cartridge sampler for collecting at least one chemical species from an environment for subsequent chemical analysis. The cartridge sampler includes a sensing unit having a body and having collection media within the body for collecting chemical samples. The collection media have at least one molecule sensor thereon for sensing molecules. The collection media are removable from the cartridge sampler. The cartridge sampler includes a sampling system adapted to sequentially sample fluid from within the environment at specified sampling times, the sampling system having a fluid inlet on an upstream side of the sensing unit, and a valve system adapted to provide fluid samples at the specified sampling times from the fluid inlet to collection media of the sensing unit. The cartridge sampler also includes a means for tagging a geospatial location and time that the fluid samples are provided to the collection media such that after the collection media are removed from the cartridge sampler, the location and time that the fluid samples are provided to the collection media can be determined.

In other aspects, the disclosure can generally relate to various systems and methods for collecting and analyzing chemical species from various environments including a subsurface formation, a pipeline, a gaseous environment, a soil or sub-slab environment, or an aqueous environment. In these aspects, the systems and methods include the use of the cartridge sampler described above fitted on or within a device designed for moving within the particular environment. The cartridge sampler is configured to sequentially sample fluid from within the particular environment as the device moves within the environment such that the collection media collects the at least one chemical species. The at least one chemical species can then be subsequently analyzed at a laboratory location.

### BRIEF DESCRIPTION OF THE DRAWINGS

These and other objects, features and advantages of the present disclosure will become better understood with reference to the following description, appended claims and accompanying drawings. The drawings are not considered limiting of the scope of the appended claims. Reference numerals designate like or corresponding, but not necessarily identical, elements. The drawings illustrate only example embodiments. The elements and features shown in the drawings are not necessarily to scale, emphasis instead being placed upon clearly illustrating the principles of the example embodiments. Additionally, certain dimensions or positionings may be exaggerated to help visually convey such principles.

FIG. 1 shows a sampler for collecting chemical species from an environment.

FIGS. 2-6 show schematic diagrams of field system in which example embodiments can be applied.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

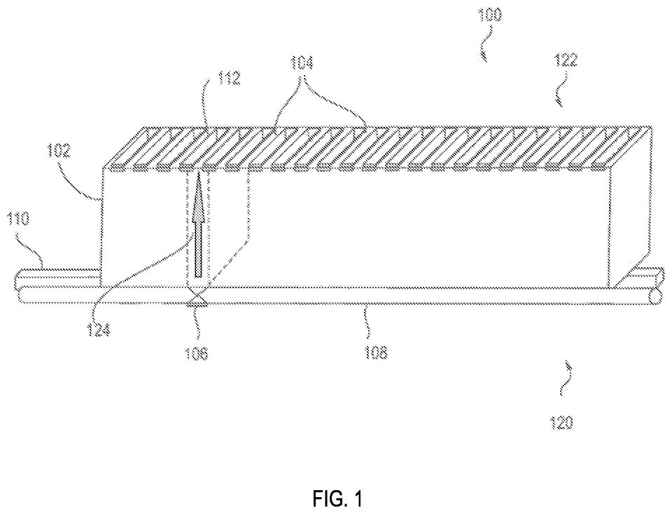

FIG. 1 depicts an embodiment of a cartridge sampler 100 for collecting chemical species from an environment for subsequent chemical analysis. The cartridge sampler 100 includes a sensing unit having a body and having collection media, also referred to as sorptive media, within the body for collecting chemical samples. The collection media have at least one molecule sensor thereon for sensing molecules. The collection media are removable from the cartridge sampler. The cartridge sampler includes a sampling system adapted to sequentially sample fluid from within the environment at specified sampling times, the sampling system having a fluid inlet on an upstream side of the sensing unit, and a valve system adapted to provide fluid samples at the specified sampling times from the fluid inlet to collection media of the sensing unit.

The illustrated cartridge sampler 100 is a sensing unit in the form of a segmented cartridge 102 (e.g., a body) having a sequence of slots 104 and collection media with a molecule sensor in each slot 104 for sensing molecules. Each slot 104 contains a molecule sensor that includes one or more removable sorptive media that may have a coating that facilitates sorption or retention of molecules from fluid that flows through the slots 104. Sorption is the process by which one substance becomes attached to another; this can take the form of absorption, adsorption or ion exchange. The sorptive media can be in the form of collection discs (no particular shape required). The sorptive media can be permeable such that fluid flows through the media, as in the form of porous filtration media which may or may not be coated to enhance collection of target molecule(s). The target molecule(s) or target species can include organic compounds, metals including mercury, inorganic compounds, microorganisms, and/or genetic material. When the target molecule(s) includes mercury, the sorptive media can be coated with a sorbent material capable of sorbing mercury, e.g., an organic or inorganic material selected from carbon, coated or activated carbon (also referred to as “treated carbon,” or carbon subjected to some kind of treatment or impregnation to enhance the collection/sorption of the target species), thiolated compounds, clay, and/or bauxite mud.

In certain embodiments, the cartridge sampler 100 includes multiple zones having differing molecule sensors on the collection media. For example, the multiple zones may correspond to different slots 104, or groups of slots 104. The use of different zones with differing molecule sensors may allow for time series sampling and detection of multiple target species (e.g., compounds, molecules, organisms).

In one embodiment, as shown in FIG. 1, fluid flows through the slots 104 in the direction indicated by arrow 124 so that the cartridge sampler 100 has a downstream surface 122 and an upstream surface 120. Cartridge sampler 100 shape and size are flexible and may be configured based on aspects of a particular sampling run. The number of slots 104 in the cartridge sampler 100 depends on various factors including but not limited to weight/size/power limitations on the device, what type of molecules are being sampled, and/or the planned time/distance of the device.

A valve system, which may include one or more movable valves 106, is used to control the flow of fluid through successive slots 104. The illustrated movable valve 106 is positioned on a transport mechanism 108 (for example a threaded shaft) on the upstream surface 120 of the cartridge sampler 100, the transport mechanism and valve 106 being controlled by one or more controllers (not shown). When the movable valve 106 opens adjacent a specific slot, fluid flows from a fluid inlet 110 through the valve 106 and then through an opened slot 112 for a specified sample time during which molecules are sensed by the molecule sensor, for example by being captured on that slot's sorptive disc. In this respect, the valve system is adapted to sequentially open the slots to provide the fluid samples at the specified sampling times from the fluid inlet.

The sample time is configurable, for example to suit the specific application (what is being sampled under which circumstances), and may depend on a number of factors including and not limited to the speed that the device is travelling at, the type of molecules being sampled, an estimated concentration of molecules in the fluid and/or the flow rate through the slot 112. For example, sampling times may be increased to reduce the number of total samples or to produce a single composite sample over a larger area. Alternatively, sampling times may be reduced to increase the sampling resolution over a smaller area. For example, the sample time may be greater than 1 second, or greater than 5 seconds, or greater than 10 second, or even greater than 20 seconds. An average fluid flow rate through the opened slot 112 may be inferred, for example in view of the speed of the device (referred to herein as the passive flow rate). The inferred flow rate is determined by a controller and stored on the controller's memory together with the log data for respective samples.

Alternatively, the inlet 110 may include a flow controller for actively controlling the fluid flow rate by pumping water through the sampler 100 and/or one or more flow meters (not shown) that may be positioned in inlet 110 to measure passive and/or active flow rate. The flow per sample period is logged. The sample represents an average over the path traveled by the sampler 100 during the sample time, and the flow rate is used to calculate the average concentration. In some embodiments the downstream surface 122 of the sampler 100 includes a cover assembly (not shown). The cover assembly includes a fluid outlet to enable removal of fluid that has passed through the opened slot 112. In some embodiments the outlet is an outlet manifold along the downstream surface of the sampler 100. In some embodiments the movable valve 106 is a valve pair, with a first valve at the upstream surface 120 and a second valve at the downstream surface 122, the valve pair moved together by the transport mechanism. The second valve facilitates the flow of fluid away from the opened slot 112 and through the fluid outlet.

In certain embodiments, the sorptive media may be in the form of a series of tubes, e.g., straw-shaped elements of the media. The tubes may be constructed to have a high surface area to volume ratio, so that the tubes act like filters. Target molecules are either sorbed to the tube walls or pass through the tube walls and are retained in material on the outside of the tubes. Additionally or alternatively, the tubes may be coated on the inside of the tubes. By way of non-limiting example, the total volume of the tubes may be about 1 to 2 L.

The sorptive media may be arranged so that the fluid being sampled flows across surfaces of the media. In such embodiments, the sorptive media may be a thin sheet of any suitable material, such as plastic, polymer or composite material that is coated with a coating designed to collect and retain the target molecule(s). For instance, the coating can be a thiol resin, polydimethylsiloxane (PDMS) or other substance designed to adhere to the sorptive media and to sorb/bond with a target species such as a hydrocarbon or metal in the fluid. Alternatively, the sorptive media may be uncoated and constructed of a sorbent material, e.g., a filter media constructed from a reactive/sorbent polymer. For instance, the filter material can be constructed from polyethylene or surface-functionalized clay, or impregnated or coated with a thiol resin or PDMS when the target molecule(s) are hydrocarbons and/or metals. The surface area of the sorptive media may be optimized for enhanced mass transfer rate of the target molecule(s) onto and/or into the media. For instance, in the case of the fluid being water and the target species being an organic substance, a sorptive media with the property of a very high organic carbon-water partition co-efficient (K_{oc}) can be used.

Many target metals are soluble under acidic conditions and precipitate under basic conditions so the sample media for metals may also be strong base to convert dissolved metals into solid phase metal oxides and remove them from the fluid and retain them in the sampler. In some embodiments, the sorptive media may be a permeable media impregnated with a strong base, e.g., sodium hydroxide, sodium bicarbonate, potassium hydroxide, etc. to precipitate the metals and then collect them. Alternatively, a strong base treatment can be placed inline upstream of the sorptive media.

In some embodiments, the target molecule(s) are environmental DNA (eDNA). Environmental DNA (eDNA) analysis is an effective method of determining the presence of aquatic organisms such as fish, amphibians, and other taxa. It is important not to degrade the eDNA before analysis. In such cases, the sorptive media can be cellulose ester filter media, polyethersulfone filter media or other filtration media designed to concentrate the eDNA material for subsequent analysis. eDNA filters may also contain a lysis buffering agent, or a lysis buffer introduced during the sampling process. Microbial filter media can be used for filtering microorganisms from the fluid being sampled. In the subsequent laboratory analysis of eDNA, polymerase chain reaction (PCR) techniques are used to grow a sample which is then compared with a library to match the DNA and identify the organism(s) present.

The shape and size of the cartridge 102 are flexible and may be configured based on aspects of a particular sampling application. For example a cartridge 102 may be shaped to maximize the number of samples and to fit within the physical constraints, or to minimize/maximize the volume and/or retention time of liquid in contact with the media. The number of slots 104 in the cartridge 102 depends on various factors including but not limited to weight/size/power limitations, what type of molecules are being sampled, and/or the planned time/distance of use. For example, the size, shape and position of the cartridge 102, slots 104 and media are all configurable, and therefore in different configurations, the sampling device may have (by way of non-limiting example) between 100 and 1000 slots, between 1000 and 4000 slots, or more than 5000 slots.

The cartridge sampler 100 also includes a means for tagging the geospatial location and time that the fluid samples are provided to the collection media such that after the collection media are removed from the cartridge sampler, the location and time that the fluid samples were provided to the collection media can be determined. For each sample, the geospatial location for the sample as well as a time of sample collection is recorded. If a sample is collected over time while the sampler is moving, then the geospatial location will actually be a vector path and the time will have a start and finish time. A geospatial positioning system (GPS) will be incorporated into whatever tool or device is deployed to carry the sampler. This spatial information can be electronically tagged and tied to the time to the sample identification number, such as by PLC to a digital data recorder. In some embodiments, an inertial measurement unit (IMU) that measures specific force and angular rate using a combination of accelerometers, gyroscopes and sometimes magnetometers can be included in the system to determine the geospatial location.

In some embodiments, the cartridge sampler 100 is fitted on or within a device designed for moving within the particular environment, enabling collecting and analyzing chemical species from various environments. The cartridge sampler 100 is subsequently recovered from the environment in order to remove the sensing unit and physically send the collection media to a laboratory for analysis of the chemical species collected. All the devices need to be retrievable in order to recover the cartridge and the collection media. The volume of the collected sample may need to be known for the subsequent laboratory analysis. The subsequent laboratory analysis determines the content of the chemical species present in the collection media and the geospatial location and time that the fluid samples were collected by the collection media.

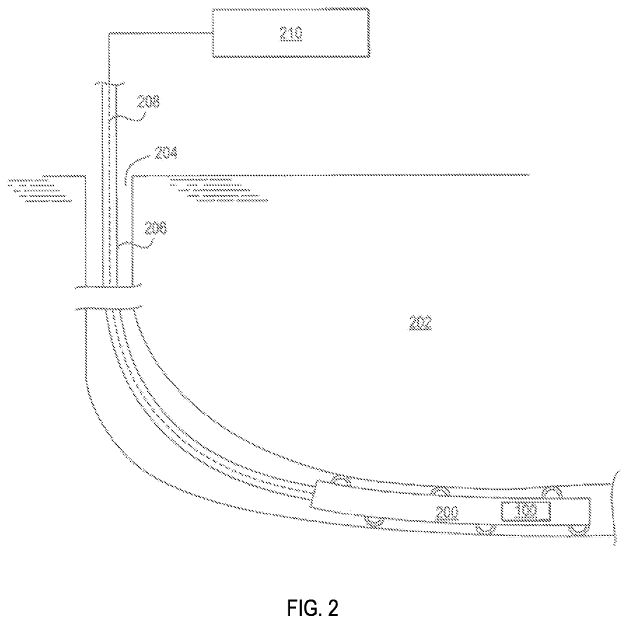

In one embodiment, referring to FIG. 2, the cartridge sampler 100 is fitted on or within a downhole tool 200 for collecting and analyzing chemical species from a subsurface formation 202. In one embodiment, the downhole tool 200 can be a downhole tractor device, e.g., disclosed in U.S. Pat. No. 6,273,189, which is incorporated by reference herein. The tractor device can be equipped with the cartridge sampler 100 for collecting chemical species in a wellbore 204. The cartridge sampler 100 sequentially samples fluid from within the wellbore 204 as the tool 200 moves within the wellbore such that the collection media collects the at least one chemical species from the fluid. In one embodiment, the downhole tool 200 can be connected to surface power and controls 210 via a wireline 208 provided in coiled tubing 206. In one embodiment, the downhole tool can be an underground robot such as that used in the proposed BADGER (“RoBot for Autonomous unDerGround trenchless opERations, mapping and navigation”) autonomous underground robotic system (under development by the BADGER Consortium and described further at www.badger-robotics.eu/). The BADGER autonomous underground robotic system is designed to drill, maneuver, localize, map and navigate in the underground space. The underground robot autonomously tunnels, navigates and executes tasks in the subsurface space. The robot can be equipped with the cartridge sampler 100 for collecting chemical species from fluid (liquid or gas) in a subsurface formation.

Understanding the mercury concentration in a well formation can be an important aspect of planning for production and site infrastructure investment. Typically, it is important to know how much mercury will be in the formation early on in the process of field evaluation before completing a well flow test (also called a daily test) as this type of test requires infrastructure such as pipelines or storage vessels to collect the well fluids when above ground. If the surface infrastructure is not in place, then it is possible to conduct a Drill Stem Test (DST) in which a portion of the well is separated with packers such that a portion of the well can be flow tested while retaining all of the well fluids within the drill stem. As a result, a very small volume of fluid can be flowed into the drill stem and recovery of this fluid for mercury testing is difficult and problematic, mainly because volumes of well fluids recovered are small and the mercury is highly reactive with the sample containers affecting the accuracy of results. A downhole well tool equipped with the cartridge sampler 102 of the present disclosure may provide a more reliable estimate of mercury in the well fluids, either as part of a DST or during a well logging exercise at the completion of well drilling.

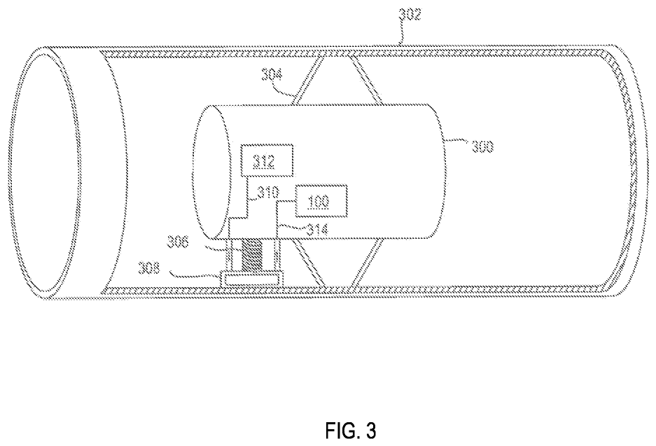

In one embodiment, referring to FIG. 3, the cartridge sampler 100 is fitted on or within a pipeline inspection tool 300 for collecting and analyzing chemical species from the interior surface of a pipeline, riser or conduit 302. The pipeline inspection tool can be any suitable pigging or pipeline inspection device onto which or in which the cartridge sampler 100 can be positioned such that fluid (liquid or gas) inside the pipeline passes onto or through the collection media. As nonlimiting examples, the pipeline inspection tool 300 can be similar to those disclosed in U.S. Pat. Nos. 4,780,962; 4,105,972; 4,123,847 and 3,576,043, each of which are incorporated by reference herein. The pipeline inspection tool 300 can be equipped with a centralizer 304. At a desired location, a sampling foot 308 is deployed against the pipeline wall. The sampling foot 308, which can be held in place by spring 306, creates a sealed space within which a fluid can be circulated from a pump or reservoir 312 within the pipeline inspection tool 300 by way of a fluid line 310, into the sampling foot 308 so that it can contact the pipeline wall. The fluid is then returned to cartridge sampler 100 within the pipeline inspection tool 300 by way of a fluid line 314.

The fluid is selected to free targeted molecules from the inside surface of the pipeline (e.g. by reaction, dissolution, desorbtion, etc.) and dissolve or entrain the molecules into the fluid. Within the cartridge sampler 100, the targeted material is collected and concentrated. The cartridge sampler 100 samples material from the interior surface of the pipeline 302 at specific locations within the pipeline such that the collection media collects at least one chemical species from the material on the inside surface of the pipeline, which can include corrosion products, gas hydrates, mercury, or other adhered, adsorbed or absorbed material on the pipeline wall. Current pig technology can move through the pipeline and use sensors to inspect pipe conditions and remove blockages; however, sampling capabilities are limited. For example, currently when it is desired to determine how much mercury has been impregnated into a section of pipeline, one must seal off a section with packers and flood the pipeline with a fluid to mobilize the mercury along the sealed length and then recover the fluid and analyze it. This test provides an averaged concentration along the length of the pipeline but this gives no information about how the concentrations may vary along the pipeline. For example, mercury may be concentrated near the inlet end of the pipeline but there is no way to determine this with the current state of testing capability. Likewise, pigs are currently very limited in their capability for collecting samples from discrete parts of the pipeline.

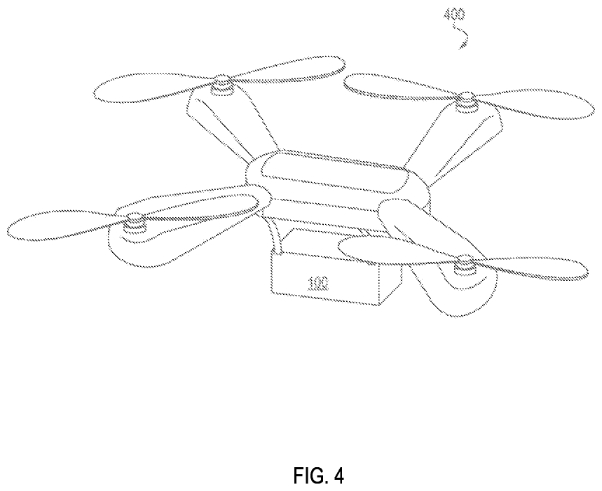

In one embodiment, referring to FIG. 4, the cartridge sampler 100 is fitted on or within a drone 400 for collecting and analyzing chemical species from a gaseous environment. The drone 400 may be any suitable device onto which or in which the cartridge sampler 100 can be positioned such that gas can pass onto or through the collection media as the drone moves through the gaseous environment. For instance, the drone can be similar to that disclosed in U.S. Pat. No. 10,095,087 or U.S. Pat. Pub. No. US20180136093, each of which are incorporated by reference herein. A qualitative gas sampler (e.g. PID or FID) can be added. GPS and sample collection time information can also be collected. Unlike known systems, in this embodiment, quantitative sampling is possible. Additionally, very small size samples can be collected enabling multiple samples to be collected and retained on a very small platform.

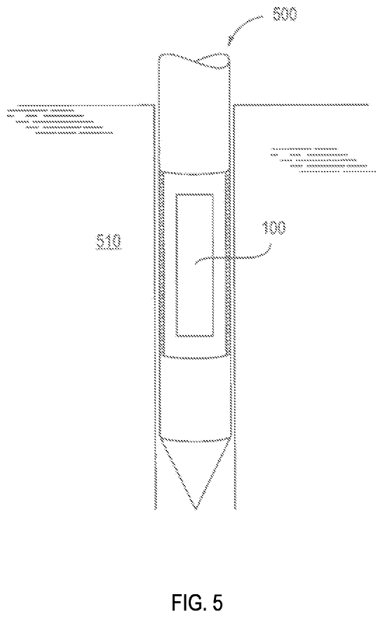

In one embodiment, referring to FIG. 5, the cartridge sampler 100 is fitted on or within a probe 500 for collecting and analyzing chemical species from a soil or sub-slab (e.g., beneath a building) environment 510. The probe 500 can be any suitable sampling probe for positioning at least partially within the soil or sub-slab environment 510 and onto/in which the cartridge sampler can be positioned. For instance, the probe can be similar to that disclosed in U.S. Pat. No. 4,804,050, which is incorporated by reference herein. Fluid, e.g., liquid, groundwater, gas and/or soil gas, can pass onto or through the collection media when the probe 500 is inserted into the soil or sub-slab environment 510. The cartridge sampler 100 samples at least one chemical species from within the soil or sub-slab environment over a period of time.

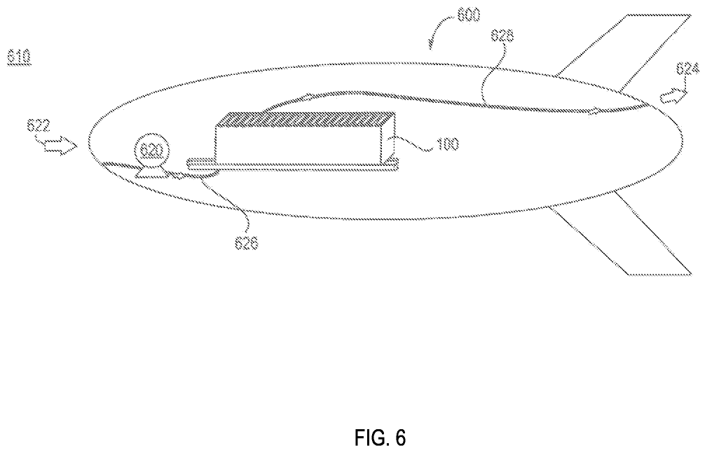

In one embodiment, referring to FIG. 6, the cartridge sampler 100 is fitted on or within a device 600 for collecting and analyzing chemical species from an aqueous environment 610. For instance, the device 600 can be similar to those disclosed in U.S. Pat. Nos. 8,265,809 and 8,352,105, each of which are incorporated by reference herein. The cartridge sampler 100 sequentially samples liquid from within the aqueous environment 610 such that the collection media collects the at least one chemical species from the aqueous environment. The cartridge sampler 100 can be removed from the device 600 and the collection media can be removed from the cartridge sampler 100 for subsequent analysis of the at least one chemical species at a laboratory location. The aqueous environment can include ocean water, ballast water in a vessel, water in the vicinity of an oil production platform or a vessel, well fluids, produced water, or storm water.

The device 600 can be any suitable device onto which or in which the cartridge sampler 100 can be fitted such that liquid can pass onto or through the collection media in the aqueous environment when the device is positioned at least partially within the aqueous environment. In some embodiments, the device 600 is dragged behind a vessel in an ocean environment to collect samples. In some embodiments, the device 600 is an ROV, an AUV or a glider in an ocean environment. For example, the device 600 can be similar to the autonomous, unmanned surface vehicle commercially available under the trade name Wave Glider™ from Liquid Robotics, Inc. (a wholly owned subsidiary of The Boeing Company). As shown, water enters an inlet 622 in the device 600 and can be directed to an optional pump 620 to deliver the water to the cartridge sampler 100 via line 626. After the fluid passes through the cartridge sampler 100 and the target molecules are collected on the collection media, the fluid is directed outside of the cartridge sampler 100 through a tube 628 and discharged back into the environment 610 through an outlet 624.

In some embodiments, the system that includes the cartridge sampler 100 also includes a real time sensor for continuously monitoring a parameter in real time, and a process controller, coordinated by a suitable programmable logic controller (PLC). The process controller determines when the parameter continuously monitored by the real time sensor has reached a predetermined triggering value. In one embodiment, in the event that the triggering value is detected, the process controller generates an alarm to alert an operator. In the event that the triggering value is detected, the process controller retrieves and uses the tagged location and time that the fluid samples corresponding to the triggering value were provided to the collection media to determine a further sampling location. The process controller can then cause the cartridge sampler 100 to collect further samples for subsequent analysis at the laboratory location. In one embodiment, the real time sensor is a subsurface sensing tool that penetrates a subsurface environment. For example, the subsurface sensing tool can be a cone penetrometer.

### ADDITIONAL DESCRIPTION

The following clauses are provided as additional description of various embodiments of the invention.

Embodiment 1. A cartridge sampler for collecting at least one chemical species from an environment for subsequent chemical analysis, the cartridge sampler comprising: (a) a sensing unit having a body and having collection media within the body for collecting chemical samples, wherein the collection media have at least one molecule sensor thereon for sensing molecules and wherein the collection media are removable from the cartridge sampler; (b) a sampling system adapted to sequentially sample fluid from within the environment at specified sampling times, the sampling system comprising a fluid inlet on an upstream side of the sensing unit, and a valve system adapted to provide fluid samples at the specified sampling times from the fluid inlet to collection media of the sensing unit; and (c) a means for tagging a geospatial location and time that the fluid samples are provided to the collection media such that after the collection media are removed from the cartridge sampler, the location and time that the fluid samples are provided to the collection media can be determined.

Embodiment 2. The cartridge sampler of embodiment 1, wherein the collection media are discs in slots in the body of the sensing unit; and the valve system is adapted to sequentially open the slots to provide the fluid samples at the specified sampling times from the fluid inlet.

Embodiment 3. The cartridge sampler of any preceding embodiment, wherein the collection media are coated with the at least one molecule sensor comprising a sorptive material adapted to retain the molecules.

Embodiment 4. The cartridge sampler of any preceding embodiment, wherein the collection media are arranged so that the fluid being sampled flows across surfaces of the collection media.

Embodiment 5. The cartridge sampler of any preceding embodiment, wherein the collection media are permeable media and the fluid being sampled flows through the collection media.

Embodiment 6. The cartridge sampler of any preceding embodiment, wherein the collection media comprise microbial filters for filtering microorganisms from the fluid being sampled.

Embodiment 7. The cartridge sampler of any preceding embodiment, wherein the sensing unit comprises multiple zones having differing molecule sensors on the collection media therein.

Embodiment 8. The cartridge sampler of any preceding embodiment, wherein the collection media collect at least one target species selected from the group consisting of organic compounds, metals, inorganic compounds, microorganisms, genetic material and combinations thereof.

Embodiment 9. The cartridge sampler of any preceding embodiment, wherein the at least one target species comprises mercury and the collection media is coated with a sorbent material capable of sorbing mercury.

Embodiment 10. The cartridge sampler of any preceding embodiment, wherein the sorbent material comprises an organic or inorganic material selected from the group consisting of carbon, treated carbon, clay, bauxite mud and combinations thereof.

Embodiment 11. The cartridge sampler of any preceding embodiment, wherein the at least one target species comprises a metal and the collection media comprise polyethylene or surface-functionalized clay.

Embodiment 12. The cartridge sampler of any preceding embodiment, wherein the at least one target species comprises a metal soluble under acidic conditions and the collection media comprise a permeable media impregnated with a strong base selected from sodium hydroxide, sodium bicarbonate, potassium hydroxide and combinations thereof.

Embodiment 13. The cartridge sampler of any preceding embodiment, wherein the at least one target species comprises a metal and the collection media is coated with a thiol resin, polydimethylsiloxane, a surface-functionalized clay and combinations thereof.

Embodiment 14. The cartridge sampler of any preceding embodiment, wherein the at least one target species comprises genetic material and the collection media is a filtration media to collect the selected from the group consisting of or other filtration media designed to concentrate the genetic material for subsequent analysis.

Embodiment 15. The cartridge sampler of any preceding embodiment, wherein the filtration media comprises cellulose ester and/or polyethersulfone filtration media.

Embodiment 16. The cartridge sampler of any preceding embodiment, wherein the filtration media comprises a lysis buffering agent.

Embodiment 17. The cartridge sampler of any preceding embodiment, wherein the at least one target species comprises a hydrocarbon and the collection media is selected from the group consisting of polyethylene, polydimethylsiloxane, surface-functionalized clay and combinations thereof.

Embodiment 18. The cartridge sampler of any preceding embodiment, wherein the at least one target species comprises a hydrocarbon and the collection media is coated with polyethylene, polydimethylsiloxane, and/or a surface-functionalized clay.

Embodiment 19. The cartridge sampler of any preceding embodiment, wherein the collection media comprise tubes of permeable media wherein the molecules are sorbed to tube walls of the tubes.

Embodiment 20. A system for collecting and analyzing chemical species from a subsurface formation, comprising: a tool for moving within a wellbore in the subsurface formation; and the cartridge sampler of any preceding embodiment fitted on or within the tool, wherein the environment is an interior of the wellbore in the subsurface formation, and the cartridge sampler is configured to sequentially sample fluid from within the wellbore as the tool moves within the wellbore such that the collection media collects the at least one chemical species from the fluid.

Embodiment 21. A system for collecting and analyzing chemical species within a pipeline, comprising: a pipeline inspection tool for moving within the pipeline; and the cartridge sampler of any preceding embodiment fitted on or within the pipeline inspection tool, wherein the environment is an interior of the pipeline, and the cartridge sampler is configured to sequentially sample fluid from the interior of the pipeline as the pipeline inspection tool moves within the pipeline such that the collection media collects at least one chemical species from the fluid.

Embodiment 22. A system for collecting and analyzing chemical species within a gaseous environment, comprising: a drone for moving within the gaseous environment; and the cartridge sampler of any preceding embodiment fitted on or within the drone, wherein the cartridge sampler is configured to sequentially sample gas from within the gaseous environment as the drone moves within the gaseous environment such that the collection media collects at least one chemical species from the gas.

Embodiment 23. A system for collecting and analyzing chemical species within a soil or sub-slab environment, comprising: a probe for positioning at least partially within the soil or sub-slab environment; and the cartridge sampler of any preceding embodiment fitted within the probe, wherein the cartridge sampler is configured to sequentially sample at least one chemical species from within the soil or sub-slab environment such that the collection media collects the at least one chemical species from the soil or sub-slab environment over a period of time.

Embodiment 24. A system for collecting and analyzing chemical species within an aqueous environment, comprising: a sampling probe for positioning at least partially within the aqueous environment; and the cartridge sampler of any preceding embodiment fitted within the sampling probe, wherein the environment is the aqueous environment, and the cartridge sampler is configured to sequentially sample liquid from within the aqueous environment such that the collection media collects the at least one chemical species from the aqueous environment; wherein the cartridge sampler can be removed from the sampling probe and the collection media can be removed from the cartridge sampler for subsequent analysis of the at least one chemical species at a laboratory location.

Embodiment 25. The system of any preceding embodiment wherein the collection media collects genetic material from the aqueous environment, further comprising a polymerase chain reaction (PCR) device to grow a sample and a library of DNA to match and identify organism(s) present in the genetic material.

Embodiment 26. The system of any preceding embodiment wherein the aqueous environment is ocean water, ballast water in a vessel, water in the vicinity of an oil production platform or a vessel, well fluids, produced water, or storm water.

Embodiment 27. The system of any preceding embodiment wherein the sampling probe is dragged behind a vessel in an ocean environment.

Embodiment 28. The system of any preceding embodiment wherein the sampling probe is positioned within an ROV, an AUV or a glider in an ocean environment.

Embodiment 29. The system of any preceding embodiment, further comprising a real time sensor for continuously monitoring a parameter in real time; and a process controller for: determining when the parameter continuously monitored by the real time sensor has a predetermined triggering value; in the event that the triggering value is detected, using a tagged location and time that the fluid samples corresponding to the triggering value were provided to the collection media to determine a further sampling location to cause the cartridge sampler to collect further samples for analysis at the laboratory location.

Embodiment 30. The system of any preceding embodiment wherein the real time sensor is a subsurface sensing tool that penetrates a subsurface environment.

Embodiment 31. The system any preceding embodiment wherein the real time sensor is a cone penetrometer.

Embodiment 32. The system of any preceding embodiment wherein, in the event that the triggering value is detected, the process controller generates an alarm to alert an operator.

Embodiment 33. A method for collecting and analyzing target species from a subsurface reservoir, comprising: deploying a tool adapted to move within a wellbore in the subsurface formation, wherein the tool comprises the cartridge sampler of any preceding embodiment fitted on or within the tool, sequentially sampling fluid from within the wellbore with the cartridge sampler as the tool moves within the wellbore, such that the collection media collects the at least one target species from the fluid; tagging a geospatial location and time that the fluid samples are collected by the collection media; retrieving the tool to a surface location; removing the collection media; and analyzing the collection media at a laboratory location to determine content of the target species present in the collection media and the geospatial location and time that the fluid samples were collected by the collection media.

Embodiment 34. The method of any preceding embodiment, wherein the collection media are discs in slots in the body of the sensing unit of the cartridge sampler of any preceding embodiment; and the valve system sequentially opens the slots to provide the fluid samples at the specified sampling times from the fluid inlet.

Embodiment 35. The method of any preceding embodiment, wherein the collection media are coated with the at least one molecule sensor comprising a sorptive material adapted to retain the molecules.

Embodiment 36. The method of any preceding embodiment, wherein the collection media are arranged so that the fluid being sampled flows across surfaces of the collection media.

Embodiment 37. The method of any preceding embodiment, wherein the collection media are permeable media and the fluid being sampled flows through the collection media.

Embodiment 38. The method of any preceding embodiment, wherein the collection media comprise microbial filters for filtering microorganisms from the fluid being sampled.

Embodiment 39. The method of any preceding embodiment, wherein the sensing unit comprises multiple zones having differing molecule sensors on the collection media therein.

Embodiment 40. The method of any preceding embodiment, wherein the collection media collect at least one target species selected from the group consisting of organic compounds, metals, inorganic compounds, microorganisms, genetic material and combinations thereof.

Embodiment 41. The method of any preceding embodiment, wherein the at least one target species comprises mercury and the collection media is coated with a sorbent material capable of sorbing mercury.

Embodiment 42. The method of any preceding embodiment, wherein the sorbent material comprises an organic or inorganic material selected from the group consisting of carbon, coated or activated carbon, clay, bauxite mud and combinations thereof.

Embodiment 43. The method of any preceding embodiment, wherein the at least one target species comprises a metal and the collection media comprise a thiol resin and/or a surface-functionalized clay.

Embodiment 44. The method of any preceding embodiment, wherein the at least one target species comprises genetic material and the collection media is a filtration media to collect the selected from the group consisting of or other filtration media designed to concentrate the genetic material for subsequent analysis.

Embodiment 45. The method of any preceding embodiment, wherein the filtration media comprises cellulose ester and/or polyethersulfone filtration media.

Embodiment 46. The method of any preceding embodiment, wherein the filtration media comprises a lysis buffering agent.

Embodiment 47. The method of any preceding embodiment, wherein the at least one target species comprises a hydrocarbon and the collection media is selected from the group consisting of polyethylene, polydimethylsiloxane, surface-functionalized clay and combinations thereof.

Embodiment 48. The method of any preceding embodiment, wherein the at least one target species comprises a hydrocarbon and the collection media is coated with polyethylene, polydimethylsiloxane, and/or a surface-functionalized clay.

Embodiment 49. The method of any preceding embodiment, wherein the collection media comprise tubes wherein the molecules are sorbed to the tubes.

Embodiment 50. A method for collecting and analyzing target species within a pipeline, comprising: deploying a pipeline inspection tool adapted to move within the pipeline, wherein the pipeline inspection tool comprises the cartridge sampler of any preceding embodiment fitted on or within the pipeline inspection tool; sequentially sampling fluid from within the pipeline with the cartridge sampler as the pipeline inspection tool moves within the pipeline, such that the collection media collects the at least one target species from the fluid; tagging a geospatial location and time that the fluid samples are collected by the collection media; retrieving the cartridge sampler from the pipeline inspection tool to a surface location; removing the collection media from the cartridge sampler; and analyzing the collection media at a laboratory location to determine content of the target species present in the collection media and the geospatial location and time that the fluid samples were collected by the collection media.

Embodiment 51. A method for collecting and analyzing target species within a gaseous environment, comprising: deploying a drone adapted to move within the gaseous environment, wherein the drone comprises the cartridge sampler of any preceding embodiment fitted on or within the drone; sequentially sampling fluid from within the gaseous environment with the cartridge sampler as the drone moves within the gaseous environment, such that the collection media collects the at least one target species from the gaseous environment; tagging a geospatial location and time that the at least one target species are collected by the collection media; retrieving the cartridge sampler from the drone; removing the collection media from the cartridge sampler; and analyzing the collection media at a laboratory location to determine content of the target species present in the collection media and the geospatial location and time that the fluid samples were collected by the collection media.

Embodiment 52. A method for collecting and analyzing target species within a soil or sub-slab environment, comprising: positioning at least partially within the soil or sub-slab environment a probe comprising the cartridge sampler of any preceding embodiment fitted therein; sequentially sampling with the cartridge sampler at least one target species from within the soil or sub-slab environment such that the collection media collects the at least one target species from liquid or gas in the soil or sub-slab environment over a period of time; tagging a geospatial location and time that the at least one target species are collected by the collection media; retrieving the cartridge sampler from the probe; removing the collection media from the cartridge sampler; and analyzing the collection media at a laboratory location to determine content of the target species present in the collection media and the geospatial location and time that the fluid samples were collected by the collection media.

Embodiment 53. A method for collecting and analyzing target species within an aqueous environment, comprising: positioning at least partially within the aqueous environment a sampling probe comprising the cartridge sampler of any preceding embodiment fitted therein; sequentially sampling with the cartridge sampler at least one target species from within the aqueous environment such that the collection media collects the at least one target species from the aqueous environment over a period of time; tagging a geospatial location and time that the at least one target species are collected by the collection media; retrieving the cartridge sampler from the sampling probe; removing the collection media from the cartridge sampler; and analyzing the collection media at a laboratory location to determine content of the target species present in the collection media and the geospatial location and time that the fluid samples were collected by the collection media.

Embodiment 54. The method of any preceding embodiment wherein the aqueous environment is ocean water, ballast water in a vessel, water in the vicinity of an oil production platform or a vessel, well fluids, produced water, or storm water.

Embodiment 55. The method of any preceding embodiment wherein the sequentially sampling comprises dragging the sampling probe behind a vessel in an ocean environment.

Embodiment 56. The method of any preceding embodiment wherein the sampling probe is positioned within a ROV, an AUV or a glider in an ocean environment.

Embodiment 57. The method of any preceding embodiment, further comprising, using a process controller: continuously monitoring a parameter in real time using a real time sensor to determine when the parameter has a predetermined triggering value; and in the event that the triggering value is detected, using a tagged location and time that the fluid samples corresponding to the triggering value were provided to the collection media to determine a further sampling location to cause the cartridge sampler to collect further samples for analysis at the laboratory location.

Embodiment 58. The method of any preceding embodiment wherein the real time sensor is a subsurface sensing tool that penetrates a subsurface environment.

Embodiment 59. The method of any preceding embodiment further comprising, in the event that the triggering value is detected, generating an alarm to alert an operator.

It should be noted that only the components relevant to the disclosure are shown in the figures, and that many other components normally part of a sampling sand sensing system are not shown for simplicity.

For the purposes of this specification and appended claims, unless otherwise indicated, all numbers expressing quantities, percentages or proportions, and other numerical values used in the specification and claims are to be understood as being modified in all instances by the term “about.” Accordingly, unless indicated to the contrary, the numerical parameters set forth in the following specification and attached claims are approximations that can vary depending upon the desired properties sought to be obtained by the present disclosure. It is noted that, as used in this specification and the appended claims, the singular forms “a,” “an,” and “the,” include plural references unless expressly and unequivocally limited to one referent.

Unless otherwise specified, the recitation of a genus of elements, materials or other components, from which an individual component or mixture of components can be selected, is intended to include all possible sub-generic combinations of the listed components and mixtures thereof. Also, “comprise,” “include” and its variants, are intended to be non-limiting, such that recitation of items in a list is not to the exclusion of other like items that may also be useful in the materials, compositions, methods and systems of this disclosure.

What is claimed is:

1. A cartridge sampler for collecting at least one chemical species from an environment for subsequent chemical analysis, the cartridge sampler comprising:
a. a sensing unit having a body and having collection media within the body for collecting chemical samples, wherein the collection media have at least one molecule sensor thereon for sensing molecules and wherein the collection media are removable from the cartridge sampler;
b. a sampling system adapted to sequentially sample fluid from within the environment at specified sampling times, the sampling system comprising a fluid inlet on an upstream side of the sensing unit, and a valve system adapted to provide fluid samples at the specified sampling times from the fluid inlet to collection media of the sensing unit; and
c. a means for tagging a geospatial location and time that the fluid samples are provided to the collection media such that after the collection media are removed from the cartridge sampler, the location and time that the fluid samples are provided to the collection media can be determined.

2. The cartridge sampler of claim 1, wherein the collection media are discs in slots in the body of the sensing unit; and the valve system is adapted to sequentially open the slots to provide the fluid samples at the specified sampling times from the fluid inlet.

3. The cartridge sampler of claim 2, wherein the collection media are coated with the at least one molecule sensor comprising a sorptive material adapted to retain the molecules.

4. The cartridge sampler of claim 3, wherein the collection media are arranged so that the fluid being sampled flows across surfaces of the collection media.

5. The cartridge sampler of claim 1, wherein the collection media are permeable media and the fluid being sampled flows through the collection media.

6. The cartridge sampler of claim 1, wherein the collection media comprise microbial filters for filtering microorganisms from the fluid being sampled.

7. The cartridge sampler of claim 1, wherein the sensing unit comprises multiple zones having differing molecule sensors on the collection media therein.

8. The cartridge sampler of claim 1, wherein the collection media collect at least one target species selected from the group consisting of organic compounds, metals, inorganic compounds, microorganisms, genetic material and combinations thereof.

9. The cartridge sampler of claim 8, wherein the at least one target species comprises mercury and the collection media is coated with a sorbent material capable of sorbing mercury.

10. The cartridge sampler of claim 10, wherein the sorbent material comprises an organic or inorganic material selected from the group consisting of carbon, treated carbon, clay, bauxite mud and combinations thereof.

11. The cartridge sampler of claim 8, wherein the at least one target species comprises a metal and the collection media comprise polyethylene or surface-functionalized clay.

12. The cartridge sampler of claim 8, wherein the at least one target species comprises a metal soluble under acidic conditions and the collection media comprise a permeable media impregnated with a strong base selected from sodium hydroxide, sodium bicarbonate, potassium hydroxide and combinations thereof.

13. The cartridge sampler of claim 8, wherein the at least one target species comprises a metal and the collection media is coated with a thiol resin, polydimethylsiloxane, a surface-functionalized clay and combinations thereof.

14. The cartridge sampler of claim 8, wherein the at least one target species comprises genetic material and the collection media is a filtration media to collect the selected from the group consisting of or other filtration media designed to concentrate the genetic material for subsequent analysis.

15. The cartridge sampler of claim 14, wherein the filtration media comprises cellulose ester and/or polyethersulfone filtration media.

16. The cartridge sampler of claim 14, wherein the filtration media comprises a lysis buffering agent.

17. The cartridge sampler of claim 8, wherein the at least one target species comprises a hydrocarbon and the collection media is selected from the group consisting of polyethylene, polydimethylsiloxane, surface-functionalized clay and combinations thereof.

18. The cartridge sampler of claim 8, wherein the at least one target species comprises a hydrocarbon and the collection media is coated with polyethylene, polydimethylsiloxane, and/or a surface-functionalized clay.

19. The cartridge sampler of claim 1, wherein the collection media comprise tubes of permeable media wherein the molecules are sorbed to tube walls of the tubes.

20. A method for collecting and analyzing chemical species from a subsurface reservoir, comprising:
a. deploying a tool adapted to move within a wellbore in the subsurface formation, wherein the tool comprises the cartridge sampler of claim 1 fitted on or within the tool,
b. sequentially sampling fluid from within the wellbore with the cartridge sampler as the tool moves within the wellbore, such that the collection media collects the at least one chemical species from the fluid;
c. tagging a geospatial location and time that the fluid samples are collected by the collection media;
d. retrieving the tool to a surface location;
e. removing the collection media; and
f. analyzing the collection media at a laboratory location to determine content of the chemical species present in the collection media and the geospatial location and time that the fluid samples were collected by the collection media.

21. A method for collecting and analyzing chemical species within a pipeline, comprising:
a. deploying a pipeline inspection tool adapted to move within the pipeline, wherein the pipeline inspection tool comprises the cartridge sampler of claim 1 fitted on or within the pipeline inspection tool;
b. sequentially sampling fluid from within the pipeline with the cartridge sampler as the pipeline inspection tool moves within the pipeline, such that the collection media collects the at least one chemical species from the fluid;
c. tagging a geospatial location and time that the fluid samples are collected by the collection media;
d. retrieving the cartridge sampler from the pipeline inspection tool to a surface location;
e. removing the collection media from the cartridge sampler; and
f. analyzing the collection media at a laboratory location to determine content of the chemical species present in the collection media and the geospatial location and time that the fluid samples were collected by the collection media.

22. A method for collecting and analyzing chemical species within an aqueous environment, comprising:
a. positioning at least partially within the aqueous environment a sampling probe comprising the cartridge sampler of claim 1 fitted therein;
b. sequentially sampling with the cartridge sampler at least one chemical species from within the aqueous environment such that the collection media collects the at least one chemical species from the aqueous environment over a period of time;
c. tagging a geospatial location and time that the at least one chemical species are collected by the collection media;
d. retrieving the cartridge sampler from the sampling probe;
e. removing the collection media from the cartridge sampler; and
f. analyzing the collection media at a laboratory location to determine content of the chemical species present in the collection media and the geospatial location and time that the fluid samples were collected by the collection media.
